(19) United States
(12) Patent Application Publication
(10) Pub. No.: US 2026/0183558 A1
(43) Pub. Date: Jul. 2, 2026

(54) SYSTEM & METHOD FOR PROVIDING PULSE ELECTROMAGNETIC FIELD (PEMF) AND SPINAL DECOMPRESSION THERAPIES

(71) Applicant: North American Medical Corporation, Marietta, GA (US)

(72) Inventors: Gidgette Becerra Rubin, Atlanta, GA (US); Carlos Becerra, Marietta, GA (US); Carlos Mario De Carro, Cartagena (CO)

(21) Appl. No.: 19/007,403

(22) Filed: Dec. 31, 2024

(51) Int. Cl. A61N 2/00; A61H 1/02; A61N 2/02

(52) U.S. Cl. CPC A61N 2/002; A61H 1/0222; A61H 1/0229; A61N 2/02; A61H 2201/0149; A61H 2201/0192; A61H 2201/10; A61H 2201/1621; A61H 2201/163; A61H 2201/165; A61H 2201/1652; A61H 2205/081

(57) ABSTRACT

Presently provided are a method and a system for pulse electromagnetic field (PEMF) and decompression therapy. The system has a treatment table or treatment chair configured and disposed to hold a first body part of a patient. A decompression harness is configured and disposed to hold a second body part of the patient, wherein at least one joint of the patient is disposed between the first body part and the second body part. The decompression harness is configured to transfer a tensional force on the at least one joint of the patient and at least one PEMF treatment module is disposed with the decompression harness and proximate the at least one joint of the patient.

### FIELD OF INVENTION

The present invention relates to systems and methods for providing pulse electromagnetic field and spinal decompression therapies.

### BACKGROUND OF THE INVENTION

Pulse Electromagnetic Field (PEMF) therapy is a type of treatment that uses low-frequency electromagnetic fields to stimulate and promote healing in the body. PEMF therapy involves the application of electromagnetic fields that pulse at controlled frequencies. These fields penetrate the body and interact with cells and tissues. The electromagnetic pulses may influence or stimulate cellular processes. For example, they may enhance cell membrane permeability, increase the production of ATP (the energy currency of cells), and improve cellular repair and regeneration. Additionally, PEMF therapy may improve blood flow or promote better circulation by dilating blood vessels, which may help with tissue repair and overall health.

PEMF therapy may be used for a variety of conditions, including pain management, bone health, muscle recovery, wound healing, and stress reduction. PEMF is often used to reduce pain and inflammation, particularly in conditions like arthritis, chronic pain, and injuries. PEMF therapy may help with bone healing and increase bone density, making it useful for fractures or osteoporosis. PEMF therapy may speed up muscle recovery and reduce soreness, accelerate wound healing, or reduce stress.

Spinal decompression therapy is a non-surgical treatment designed to relieve pressure on the spine and its associated nerves. This pressure may be caused by conditions like herniated discs, bulging discs, or spinal stenosis. The therapy may provide more space within the spinal column and alleviate symptoms such as pain, numbness, and tingling.

The most common form of spinal decompression therapy involves a specialized table or device. The patient lies on the table, and it applies gentle, controlled traction to the spine. This traction creates a negative pressure within the intervertebral discs, which may help retract herniated or bulging discs and relieve pressure on nerve roots.

By reducing pressure on the spinal discs and nerves, decompression therapy may help alleviate pain associated with conditions like herniated or bulging discs, sciatica, and spinal stenosis. The therapy may promote better nutrient flow to the spinal discs, which might aid in their healing and reduce symptoms related to disc degeneration.

Patients may experience improved spinal mobility and function following decompression therapy, which may improve overall quality of life and ability to perform daily activities.

Spinal decompression therapy typically involves a series of sessions, each lasting 15-30 minutes. The number of sessions required varies depending on the individual's condition and response to treatment.

It may be desirous to have a system configured for providing both pulse electromagnetic field and spinal decompression therapies.

### SUMMARY

In one aspect of the present disclosure, a pulse electromagnetic field (PEMF) and decompression system has a treatment table or treatment chair configured and disposed to hold a first body part of a patient and a decompression harness configured and disposed to hold a second body part of the patient. At least one joint of the patient is disposed between the first body part and the second body part. The decompression harness is configured to transfer a tensional force on the at least one joint of the patient. A tensioner link extends from the decompression harness, the tensioner link is configured and disposed to provide a decompression force to the patient. At least one PEMF treatment module is disposed with the decompression harness and proximate the at least one joint of the patient.

In another aspect of the present disclosure, a method of providing PEMF and decompression therapies to a patient is provided. The method comprises holding a first body part of a patient on a treatment table or treatment chair; disposing a PEMF treatment module with a decompression harness; holding a second body part of the patient with the decompression harness; wherein the PEMF module is disposed proximate at least one joint of the patient between the patient's first body part and the patient's second body part; tensioning the decompression harness and providing the decompression therapy to the at least one joint; activating the PEMF module and providing the PEMF therapy to the at least one joint; and removing the patient from the PEMF module and the treatment table or the treatment chair.

### BRIEF DESCRIPTION OF THE DRAWINGS

The foregoing and other features of this disclosure will become more fully apparent from the following description and appended claims, taken in conjunction with the accompanying drawings and examples. Understanding that these drawings depict only several embodiments in accordance with the disclosure and are therefore, not to be considered limiting of its scope, the disclosure will be described with additional specificity and detail through use of the accompanying drawings.

The following figures, which are idealized, are not to scale and are intended to be merely illustrative of aspects of the present disclosure and non-limiting. In the drawings, like elements may be depicted by like reference numerals. The drawings are briefly described as follows:

FIG. 1 shows an illustrative example of the presently disclosed system for providing pulse electromagnetic field (PEMF) and decompression therapy;

FIG. 2 shows an illustrative example of a decompression harness holding a body part of a patient;

FIGS. 3 and 4 show a front view of the decompression harness shown in FIG. 2;

FIG. 5 shows the decompression harness shown in FIG. 2 in an open orientation;

FIG. 6 shows a pocket in the decompression harness disposing a PEMF treatment module;

FIG. 7 is a rear view of a portion of the decompression harness shown in FIG. 2;

FIGS. 8 and 9 are front and rear views of another embodiment of a decompression harness of the present disclosure.

### DETAILED DESCRIPTION OF THE INVENTION

The presently disclosed system may provide for decompression modalities, muscular elongation, spinal mobilization and PEMF therapies.

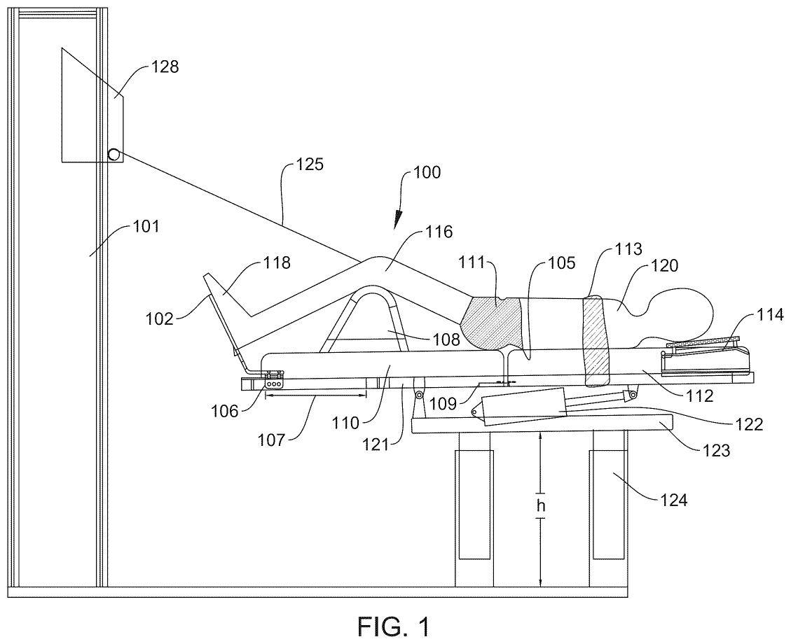

FIG. 1 shows an illustrative example of the presently disclosed system 100 for providing pulse electromagnetic field (PEMF) and decompression therapy. System 100 may have a treatment chair or treatment table 121. Treatment table 121 may be configured to be disposed in varying degree of inclinations. FIG. 1 depicts patient 120 in a substantially horizontal orientation, which may maximize a decompression therapy. Treatment table 121 may have a split-table design that separates about the patient's 120 spine to facilitate decompression modalities, muscular elongation, spinal mobilization. For example, table 121 may comprise a first portion 110 and a second portion 112. First portion 110 and second portion 112 may be separated from one another, as designated with separation 109. Separation 109 may be expanded for spinal decompression. A combination of inclination of table 121, approaching horizontal, and separation 109 may be adjusted to provide a desired degree of spinal decompression.

A desired degree of spinal decompression may also be obtained with belt or strap 113 holding the torso of patient 120 to the second portion 112 of table 121 and applying force to decompression harness 111. For example, decompression harness 111 may be secured about the pelvis of patient 120 and a tensile force may be placed on tensioner link 125 with decompression head 128. Decompression head 128 may be configured and disposed to place tension on tensioner link 125 and to move up and down on riser 101 to provide a desired angle of tension on decompression harness 111. Knee cushion 108 may be placed under knees 116, which may also aid in providing a desired degree of decompression. Head rest 114 may be positioned on table 121.

Decompression harness 111 may be configured to hold a PEMF treatment module 105 for PEMF therapy. For example, decompression harness 111 may have a pocket or other configuration for holding PEMF treatment module 105 proximate at least one joint of patient 120. For example, decompression harness 111 may be in the form of a pelvic harness and treatment module 105 may be held proximate patient's 120 back or vertebras.

System 100 may have actuator 122 configured and disposed to adjust the inclination of table 121. For example, table 121 may be pivotally held to stand 123 with pivot joint 126. For example, actuator 122 may be extended and retracted to provide a desired inclination of table 121.

System 100 may have an adjustable support 124 configured and disposed to adjust a height (h) of stand 123. It may be desired to increase or decrease height (h) for different heights of patient 120. For example, it may be desired to have a greater height (h) for a taller patient 120 and a greater extension of mounting plate 102, extended about movement arrow 107. In this embodiment, mounting plate 102 may be placed proximate the floor for easing the mounting of patient 120 onto mounting plate 102 and table 121, upon table 121 being disposed in a substantially fully inclined orientation.

In at least one embodiment, pulse electromagnetic field (PEMF) and decompression system 100 has a treatment table or treatment chair 121 configured and disposed to hold a first body part of patient 120 and a decompression harness 111 configured and disposed to hold a second body part of patient 120. At least one joint, such as one or more vertebra, of patient 120 is disposed between the first body part and the second body part. For example, the first body part may be torso and the second body part may be a pelvis. Decompression harness 111 is configured to transfer a tensional force on the at least one joint of patient 120. Tensioner link 125 extends from the decompression harness 111 and is configured and disposed to provide a decompression force to patient 120. At least one PEMF treatment module 105 is disposed with decompression harness 111 and proximate the at least one joint of patient 120.

In at least one other embodiment, PEMF and decompression system 100 has a decompression head 128 holding a terminal end of tensioner link 125. Decompression head 128 is configured and disposed to move and to increase and decrease the decompression force about the at least one joint.

In at least one additional embodiment, decompression system 100 has a harness, a belt, or other device 113 configured and disposed to hold the first body part of patient 120 with respect to treatment table or treatment chair 121.

In at least one further embodiment, treatment table or treatment chair 121 comprises a first portion 110 configured and disposed to support the first body part of patient 120 and a second portion 112 configured and disposed to support the second body part of the patient 120, the system is configured to move first portion 110 and the second portion 112 of treatment table or treatment chair 121 apart and to induce a tensional force on the patient's 120 at least one joint.

In at least one embodiment, PEMF and decompression system 100 has an actuator 122 configured and disposed to adjust an inclination of the treatment table or treatment chair 121.

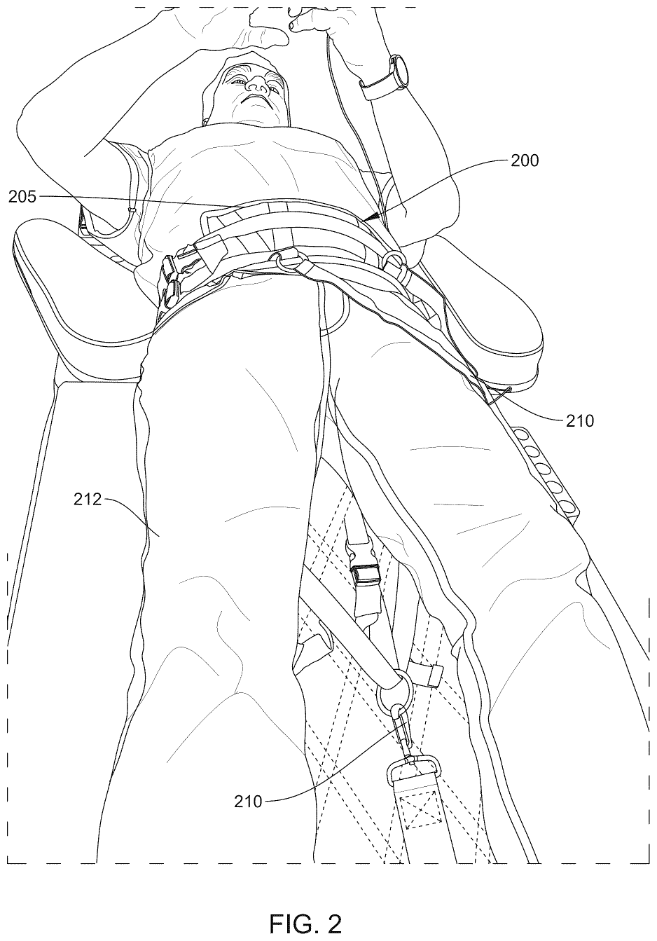

FIG. 2 shows an illustrative example of a decompression harness 200 holding a body part of a patient 212. For example, decompression harness 200 may be configured to hold patient's 212 pelvis. It is to be understood that decompression harness 200 may have a variety of configurations for holding a variety of body parts of a patient. For example, a treatment table or treatment chair may be configured and disposed to hold a first body part of a patient and a decompression harness may be configured and disposed to hold a second body part of the patient, wherein at least one joint of the patient is disposed between the first body part and the second body part. A first portion 205 of decompression harness 200 is shown disposed on the front of patient 212 and tensioner link 210 is shown extending from decompression harness 200.

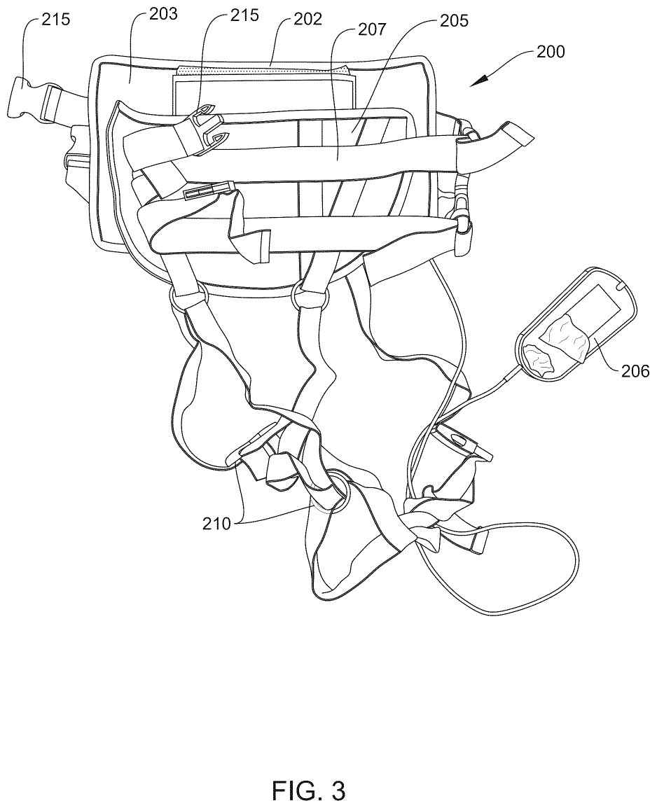

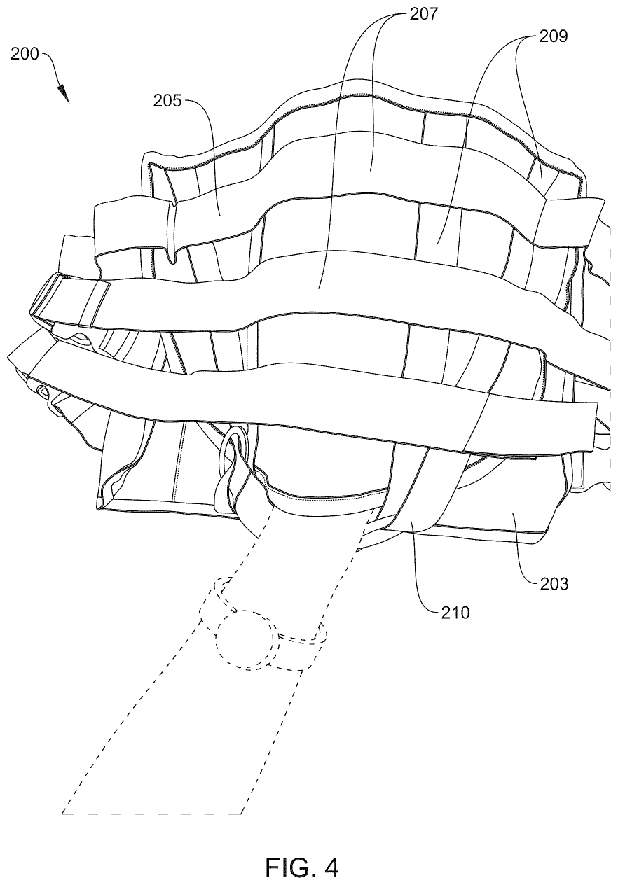

FIGS. 3 and 4 show a front view of the decompression harness 200. Decompression harness 200 may have a front portion 205 and a rear portion 203. Straps 207 are disposed on front portion 205 to extend laterally about patient 212 and may have clips 215 configured to join with lateral extending straps 211 on rear portion 203 Rear portion 203 has a pocket 202 configured for holding a PEMF treatment module 204 and disposing the PEMF treatment module proximate at least one joint of patient 212. Tensioner link 210 extends from decompression harness 200. Control 206 may provide for the control of a PEMF treatment module disposed in decompression harness 200.

In at least one embodiment, PEMF and decompression system 100 has a decompression harness 200 comprising a plurality of straps, 207 and 211, configured to be disposed laterally about the patient's 212 second body part. In at least one other embodiment, PEMF and decompression system 100 has decompression harness 200 comprising a plurality of straps, 209 and 213, configured to be disposed longitudinally about patient's 212 second body part. The plurality of straps 209 and 213, configured to be disposed longitudinally about the patient's 212 second body part comprise straps angled with respect to tensioner link 210.

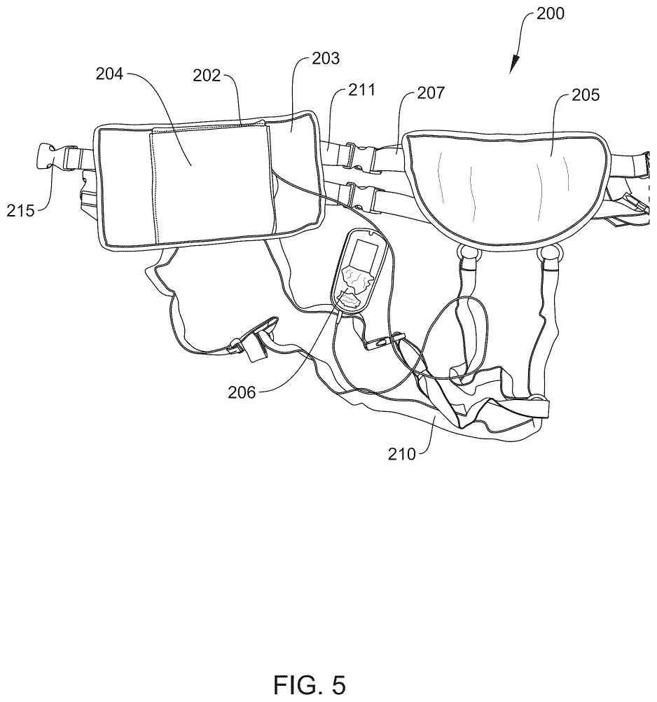

FIG. 5 shows the decompression harness 200 in an open orientation. As shown therein, laterally disposed straps 211 and 209 are disposed with front portion 205 and rear portion 204. Straps 211 and 209 are configured to extend around patient 212 and may have clips 215 configured to join straps 211 and 209 together to hold the patient's 212 second body part, or pelvis in this example. Rear portion 203 has pocket 202 configured to hold PEMF treatment module 204.

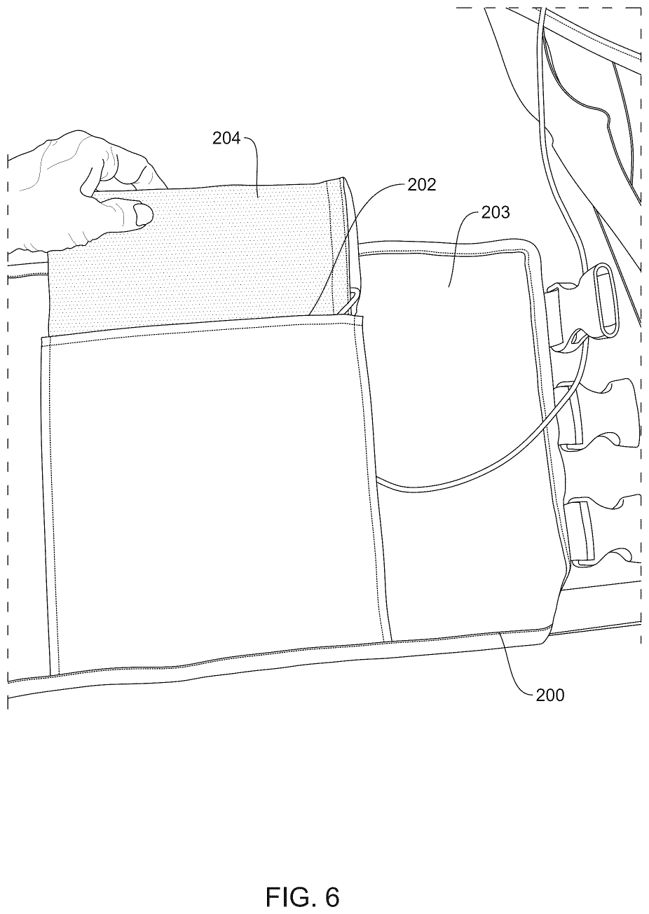

FIG. 6 shows pocket 202 in decompression harness 200 for disposing PEMF treatment module 204. In this example, PEMF treatment module 204 may be slid into pocket 202 for holding PEMF treatment module 204 with rear portion 203 of decompression harness 200.

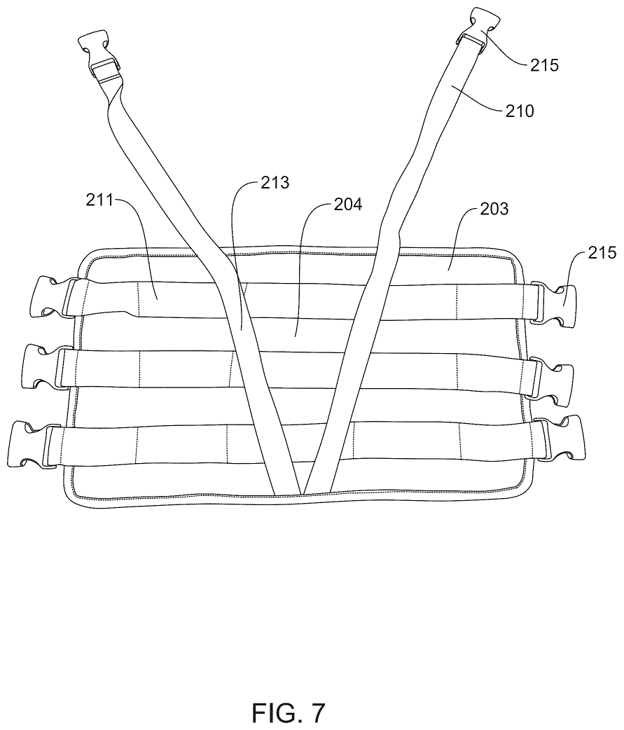

FIG. 7 is a rear view of rear portion 203 of the decompression harness 200. As shown, a plurality of straps 211 may extend laterally with rear portion 203 which may have clips 215 for joining with straps 207 on front portion 205. Longitudinally extending straps 213 may extend to, or be a part of, tensioner link 210. For example, a plurality of straps 213 may be configured to be disposed longitudinally about the patient's second body part and may be angled.

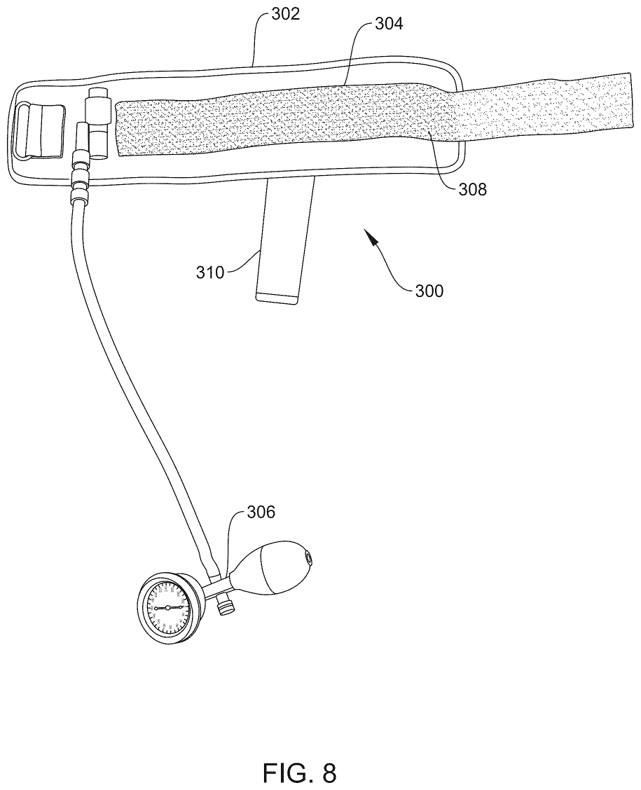

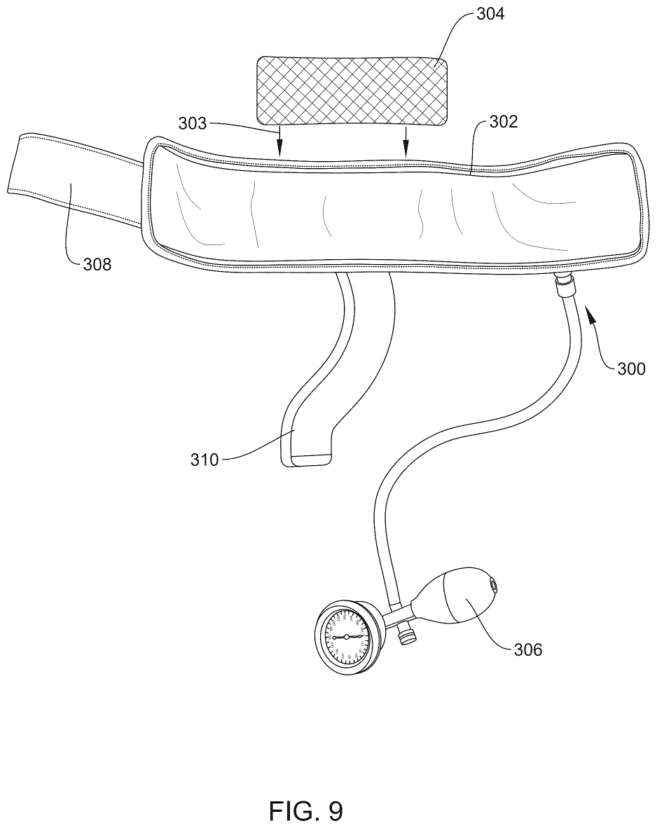

FIGS. 8 and 9 are front and rear views of decompression harness 300. Decompression harness 300 is configured and disposed to hold a body part of the patient 212. For example, decompression harness 300 may be configured to hold a wrist. Another body part of patient, for example a forearm, may be held with a treatment table or treatment chair. Decompression harness 300 and a treatment table or chair are configured to dispose at least one joint of patient 212 there between. Decompression harness 300 is configured to transfer a tensional force on the at least one joint of patient 212 disposed between the body parts held with the treatment table or treatment chair and decompression harness 300. The tensional force may be applied with tensioner link 310 extending from decompression harness 300. Tensioner link 310 is configured and disposed to provide a decompression force to patient 212. Decompression harness 300 may have an opening or pocket 302 configured for holding PEMF treatment module 304 therewith. For example, PEMF treatment module 304 may be placed in pocket 302 as shown with insertion arrows 303. Decompression harness 300 is configured to disposed at least one PEMF treatment module 304 proximate at least one joint of the patient, upon holding a body part with decompression harness 300. Decompression harness 300 may have a bladder with pump 306 for securing about patient's 212 body part. A fastener 308 may be disposed with decompression harness 300 for fastening decompression harness 300 on patient's 212 body part and holding the body part.

Presently disclosed is a method of providing PEMF and decompression therapies to a patient. The method comprises: holding a first body part of a patient on a treatment table or treatment chair; disposing a PEMF treatment module with a decompression harness; and holding a second body part of the patient with the decompression harness. The PEMF module is disposed proximate at least one joint of the patient between the patient's first body part and the patient's second body part. Tensioning the decompression harness provides the decompression therapy to the at least one joint and activating the PEMF module and provides the PEMF therapy to the at least one joint. Upon completing the therapy, the patient is removed from the decompression harness and the treatment table or the treatment chair.

The tensioning of the decompression harness and the activating the at least one PEMF may be performed simultaneously for an instance of time. Alternatively, the tensioning of the decompression harness and the activating the at least one PEMF may be performed at different times.

The PEMF module may be placed into a pocket in the decompression harness or otherwise held with the decompression harness. The tensioning of the decompression harness may be performed with the tensioning of a tensioner link extending from the decompression harness. For example, a decompression head may be moved to increase or decrease the tensioning or the changing of an angle of the applied tension of the decompression harness.

Tensioning may also be provided, or increased, by moving a first portion of the treatment table or treatment chair, holding the first body part of the patient, apart from a second portion of the treatment table or treatment chair, wherein the second portion of the treatment table or treatment chair is supporting the second body of the patient. The holding the first body part of the patient may comprise holding a torso of the patient on the treatment table or treatment chair and the holding the second body part of the patient may comprise holding a pelvis of the patient with the decompression harness.

In at least one embodiment, a pulse electromagnetic field (PEMF) and decompression system is provided. The system comprises a treatment table or treatment chair configured and is disposed to hold a first body part of a patient. A decompression harness is configured and disposed to hold a second body part of the patient, wherein at least one joint of the patient is disposed between the first body part and the second body part. The decompression harness is configured to transfer a tensional force on the at least one joint of the patient. A tensioner link extends from the decompression harness, the tensioner link is configured and disposed to provide a decompression force to the patient. At least one PEMF treatment module is disposed with the decompression harness and proximate the at least one joint of the patient.

In at least one other embodiment, the PEMF and decompression system has the decompression harness comprising a pelvic harness configured and disposed to hold a pelvis of the patient and the at least one joint of the patient comprises a lower spinal area of the patient.

One feature or aspect of an embodiment is believed at the time of the filing of this patent application to possibly reside broadly in the PEMF and decompression system, wherein the decompression harness comprises at least one pocket and is configured to dispose the at least one PEMF treatment module proximate the at least one joint of the patient.

Another feature or aspect of an embodiment is believed at the time of the filing of this patent application to possibly reside broadly in the PEMF and decompression system, further comprising a decompression head holding a terminal end of the tensioner link, the decompression head is configured and disposed to move and to increase and decrease the decompression force about the at least one joint.

Yet another feature or aspect of an embodiment is believed at the time of the filing of this patent application to possibly reside in the PEMF and decompression system, further comprising a harness, a belt, or other device configured and disposed to hold the first body part of the patient with respect to the treatment table or treatment chair.

Still another feature or aspect of an embodiment is believed at the time of the filing of this patent application to possibly reside broadly the PEMF and decompression system, wherein the treatment table or treatment chair comprises a first portion configured and disposed to support the first body part of the patient and a second portion configured and disposed to support the second body part of the patient, the system is configured to move the first portion and the second portion of the treatment table or treatment chair apart and to induce a tensional force on the patient's at least one joint.

A further feature or aspect of an embodiment is believed at the time of the filing of this patent application to possibly reside broadly the PEMF and decompression system, further comprising an actuator configured and disposed to adjust an inclination of the treatment table or treatment chair.

Another feature or aspect of an embodiment is believed at the time of the filing of this patent application to possibly reside broadly in the PEMF and decompression system, wherein the decompression harness comprises a plurality of straps configured to be disposed laterally about the patient's second body part.

Yet another feature or aspect of an embodiment is believed at the time of the filing of this patent application to possibly reside broadly the PEMF and decompression system, wherein the decompression harness comprises a plurality of straps configured to be disposed longitudinally about the patient's second body part.

Still another feature or aspect of an embodiment is believed at the time of the filing of this patent application to possibly reside broadly in the PEMF and decompression system, wherein the plurality of straps configured to be disposed longitudinally about the patient's second body part comprise straps angled toward the tensioner link.

A further feature or aspect of an embodiment is believed at the time of the filing of this patent application to possibly reside broadly in a method of providing PEMF and decompression therapies to a patient comprising the steps of: holding a first body part of a patient on a treatment table or treatment chair; disposing a PEMF treatment module with a decompression harness; holding a second body part of the patient with the decompression harness; wherein the PEMF module is disposed proximate at least one joint of the patient between the patient's first body part and the patient's second body part; tensioning the decompression harness and providing the decompression therapy to the at least one joint; activating the PEMF module and providing the PEMF therapy to the at least one joint; and removing the patient from the PEMF module and the treatment table or the treatment chair.

Another feature or aspect of an embodiment is believed at the time of the filing of this patent application to possibly reside broadly in the method, wherein the tensioning of the decompression harness and the activating the at least one PEMF are performed simultaneously for an instance of time.

Yet another feature or aspect of an embodiment is believed at the time of the filing of this patent application to possibly reside broadly in the method, wherein the tensioning of the decompression harness and the activating the at least one PEMF are performed at different times.

One feature or aspect of an embodiment is believed at the time of the filing of this patent application to possibly reside broadly the method, further comprising placing the PEMF module into a pocket in the decompression harness.

Another feature or aspect of an embodiment is believed at the time of the filing of this patent application to possibly reside broadly in the method, wherein the tensioning of the decompression harness is performed with the tensioning of a tensioner link extending from the decompression harness.

Yet another feature or aspect of an embodiment is believed at the time of the filing of this patent application to possibly reside broadly in the method, further comprising moving a decompression head and increasing or decreasing the tensioning or changing an angle of the applied tension of the decompression harness.

Still another feature or aspect of an embodiment is believed at the time of the filing of this patent application to possibly reside broadly in the method, further comprising moving a first portion of the treatment table or treatment chair, holding the first body part of the patient, apart from a second portion of the treatment table or treatment chair, wherein the second portion of the treatment table or treatment chair is supporting the second body of the patient.

A further feature or aspect of an embodiment is believed at the time of the filing of this patent application to possibly reside broadly in the method, wherein the holding the first body part of the patient comprises holding a torso of the patient on the treatment table or treatment chair and the holding the second body part of the patient comprises holding a pelvis of the patient with the decompression harness.

Nomenclature

| | |
|---|---|
| pulse electromagnetic field (PEMF) | 100 |
| and decompression system | |
| riser | 101 |
| mounting plate | 102 |
| PEMF treatment module | 105 |
| slide mount | 106 |
| movement arrow | 107 |
| knee cushion | 108 |
| separation | 109 |
| a first portion | 110 |
| decompression or pelvic harness | 111 |
| second portion | 112 |
| belt or strap | 113 |
| head rest | 114 |
| knees | 116 |
| feet | 118 |
| patient | 120 |
| table | 121 |
| actuator | 122 |
| stand | 123 |
| adjustable support | 124 |
| tensioner link | 125 |
| decompression head | 128 |
| decompression harness | 200 |
| at least one pocket | 202 |
| rear portion of decompression harness | 203 |
| PEMF treatment module | 204 |
| front portion of decompression harness | 205 |
| PEMF treatment module controller | 206 |
| straps, front latitudinal | 207 |
| straps, front longitudinal | 209 |
| tensioner link | 210 |
| straps, rear latitudinal | 211 |
| patient | 212 |
| straps, rear longitudinal | 213 |
| clip | 215 |
| decompression harness | 300 |
| at least one pocket | 302 |
| insertion arrows | 303 |
| PEMF module | 304 |
| bladder hand pump | 306 |
| fastener | 308 |
| tensioner link | 310 |

It will be understood that the examples of patents, published patent applications, and other documents which are included below in this application and which are referred to in paragraphs which state “Some examples of . . . which may possibly be used in at least one possible embodiment of the present application . . . ” may possibly not be used or useable in any one or more embodiments of the application. These references, or portions thereof, are hereby incorporated by reference herein. The purpose of incorporating U.S. patents, foreign patents, publications, etc. is solely to provide additional information relating to technical features of one or more embodiments, which information may not be completely disclosed in the wording in the pages of this application. Words relating to the opinions and judgments of the author and not directly relating to the technical details of the description of the embodiments therein are not incorporated by reference. The words all, always, absolutely, consistently, preferably, guarantee, particularly, constantly, ensure, necessarily, immediately, endlessly, avoid, exactly, continually, expediently, need, must, only, perpetual, precise, perfect, require, requisite, simultaneous, total, unavoidable, and unnecessary, or words substantially equivalent to the above-mentioned words in this sentence, when not used to describe technical features of one or more embodiments, are not considered to be incorporated by reference herein.

Some examples of features which may possibly be utilizable by at least one possible embodiment may possibly be found in the following which are incorporated by reference herein: U.S. Pat. No. 8,079,971, titled “Spinal Mobilization Treatment System with Multi-Dimensional Force Application”, filed 2009 May 29, by Carlos Becerra; U.S. Pat. No. 6,152,950, titled “Apparatus for Therapeutic Treatment of Low Back Pain”, filed 1998 Mar. 31, by Norman et al.; U.S. Ser. No. 15/627,870, titled “Spine Alignment And Decompression Systems”, by Sepulveda, Homero, filed 2017-06-2020; US20140018712, titled “Vibrating Massage Table” by Bouchet and Pageot, filed 2014-01-2016; US20230172784, titled “System & Method for Providing Whole Body Vibration Therapy and Physiotherapeutic Spinal Mobilization and Decompression Modalities”, by Rubin et al., filed Dec. 2, 2022; and U.S. Pat. No. 10,898,708, titled “System and method for applying pulsed electromagnetic fields”, by Franco-obregon et al., filed May 5, 2016

1. A pulse electromagnetic field (PEMF) and decompression system comprising:
a treatment table or treatment chair configured and disposed to hold a first body part of a patient;
a decompression harness configured and disposed to hold a second body part of the patient, wherein at least one joint of the patient is disposed between the first body part and the second body part;
the decompression harness is configured to transfer a tensional force on the at least one joint of the patient;
a tensioner link extending from the decompression harness, the tensioner link is configured and disposed to provide a decompression force to the patient; and
at least one PEMF treatment module disposed with the decompression harness and proximate the at least one joint of the patient.

2. The PEMF and decompression system of claim 1, wherein the decompression harness comprises a pelvic harness configured and disposed to hold a pelvis of the patient and the at least one joint of the patient comprises a lower spinal area of the patient.

3. The PEMF and decompression system of claim 1, wherein the decompression harness comprises at least one pocket and is configured to dispose the at least one PEMF treatment module proximate the at least one joint of the patient.

4. The PEMF and decompression system of claim 1, further comprising a decompression head holding a terminal end of the tensioner link, the decompression head is configured and disposed to move and to increase and decrease the decompression force about the at least one joint.

5. The PEMF and decompression system of claim 1, further comprising a harness, a belt, or other device configured and disposed to hold the first body part of the patient with respect to the treatment table or treatment chair.

6. The PEMF and decompression system of claim 1, wherein the treatment table or treatment chair comprises a first portion configured and disposed to support the first body part of the patient and a second portion configured and disposed to support the second body part of the patient, the system is configured to move the first portion and the second portion of the treatment table or treatment chair apart and to induce a tensional force on the patient's at least one joint.

7. The PEMF and decompression system of claim 1, further comprising an actuator configured and disposed to adjust an inclination of the treatment table or treatment chair.

8. The PEMF and decompression system of claim 1, wherein the decompression harness comprises a plurality of straps configured to be disposed laterally about the patient's second body part.

9. The PEMF and decompression system of claim 1, wherein the decompression harness comprises a plurality of straps configured to be disposed longitudinally about the patient's second body part.

10. The PEMF and decompression system of claim 9, wherein the plurality of straps configured to be disposed longitudinally about the patient's second body part comprise straps angled.

11. A method of providing PEMF and decompression therapies to a patient comprising the steps of:
holding a first body part of a patient on a treatment table or treatment chair;
disposing a PEMF treatment module with a decompression harness;
holding a second body part of the patient with the decompression harness;
wherein the PEMF module is disposed proximate at least one joint of the patient between the patient's first body part and the patient's second body part;
tensioning the decompression harness and providing the decompression therapy to the at least one joint;
activating the PEMF module and providing the PEMF therapy to the at least one joint; and
removing the patient from the decompression harness and the treatment table or the treatment chair.

12. The method of claim 11, wherein the tensioning of the decompression harness and the activating the at least one PEMF are performed simultaneously for an instance of time.

13. The method of claim 11, wherein the tensioning of the decompression harness and the activating the at least one PEMF are performed at different times.

14. The method of claim 11, further comprising placing the PEMF module into a pocket in the decompression harness.

15. The method of claim 11, wherein the tensioning of the decompression harness is performed with the tensioning of a tensioner link extending from the decompression harness.

16. The method of claim 15, further comprising moving a decompression head and increasing or decreasing the tensioning or changing an angle of the applied tension of the decompression harness.

17. The method of claim 11, further comprising moving a first portion of the treatment table or treatment chair, holding the first body part of the patient, apart from a second portion of the treatment table or treatment chair, wherein the second portion of the treatment table or treatment chair is supporting the second body of the patient.

18. The method of claim 11, wherein the holding the first body part of the patient comprises holding a torso of the patient on the treatment table or treatment chair and the holding the second body part of the patient comprises holding a pelvis of the patient with the decompression harness.
